(19) United States
(12) Patent Application Publication
(10) Pub. No.: US 2024/0099687 A1
(43) Pub. Date: Mar. 28, 2024

(54) HEMODYNAMIC MONITORING SYSTEM IMPLEMENTING ULTRASOUND IMAGING SYSTEMS AND MACHINE LEARNING-BASED IMAGE PROCESSING TECHNIQUES

(71) Applicant: ImaCor, Inc., Jericho, NY (US)

(72) Inventors: Scott L. Roth, Palm Beach Garden, FL (US); Jenn Kujawski, Northport, NY (US); Rich Lanzillotto, Medford, NY (US); Thomas Williams, Malverne, NY (US)

(21) Appl. No.: 18/273,104

(22) PCT Filed: Jan. 19, 2022
(86) PCT No.: PCT/US2022/012970

§ 371 Date: Jul. 19, 2023

Related U.S. Application Data
(60) Provisional application No. 63/139,236, filed on Jan. 19, 2021.

(51) Int. Cl. A61B 8/08; A61B 8/00; A61B 8/12; G06T 7/10; G16H 30/40; G16H 40/63

(52) U.S. Cl. CPC A61B 8/0883; A61B 8/12; A61B 8/463; A61B 8/5223; G06T 7/10; G16H 30/40; G16H 40/63; G06T 2207/10132; G06T 2207/20081; G06T 2207/20084; G06T 2207/30048

(57) ABSTRACT

A hemodynamic monitoring system comprising an ultrasound system comprising a transesophageal ultrasound probe and a computer system coupled to the ultrasound system. The computer system can be configured to calculate an image quality parameter and/or a hemodynamic parameter by segmenting images obtained via the hemodynamic monitoring system to identify a selected anatomical structure therein. The image quality and hemodynamic parameters can be displayed to users, such as medical staff, in connection with the ultrasound images.

### PRIORITY

The present application claims priority to U.S. Provisional Patent Application No. 63/139,236, titled HEMODYNAMIC MONITORING SYSTEM IMPLEMENTING ULTRASOUND IMAGING SYSTEMS AND MACHINE LEARNING-BASED IMAGE PROCESSING TECHNIQUES, filed Jan. 19, 2021, which is hereby incorporated by reference herein in its entirety.

### BACKGROUND

Everything in the ICU is focused upon maintaining patients' organ function and perfusion. Medical staff generally use hemodynamic parameters, including pressure and volume, as their guideposts. In particular, ICU nurses are specifically in charge of monitoring blood pressure and typically move to invasive arterial pressure monitoring (“A-line”) for their sicker ICU patients. Their next steps beyond monitoring blood pressure consist of “hemodynamic monitoring.” Initially the Swan-Ganz catheter defined the space with pressure and flow parameters (i.e., cardiac output and filling pressures) via invasive monitoring. Subsequently, noninvasive tools have become prominent, including cardiac output monitors. However, without the ability to accurately reproduce pressure and flow measurements, many noninvasive tools have instead focused on volume responsiveness of the heart. Despite the availability of new tools, the bottom line is that pressure and flow are the key cardiac metrics that nurses use in monitoring patients.

In sum, the Swan-Ganz catheter can be used to obtain quantitative measurements or estimates for key hemodynamic parameters, but it is invasive and its invasiveness makes it potentially dangerous for patients. Therefore, the Swan-Ganz catheter has significant drawbacks and is thus not desirable to use. In contrast, noninvasive hemodynamic monitoring tools can be beneficial compared to the Swan-Ganz catheter because they are less dangerous to patients, but noninvasive hemodynamic tools do not provide the better (or even the same) quality of output that can be obtained using the Swan-Ganz catheter. In particular, noninvasive cardiac output monitoring tools are not able to provide a better output or estimate of cardiac filling parameters than Swan-Ganz catheter. Therefore, practitioners have to make a choice between invasiveness and the quality of the output when seeking to monitor a patient's hemodynamic parameters because none of the options currently available to practitioners are able to noninvasively provide them with the highest quality hemodynamic output in a noninvasive manner. It is highly desirable to address the issue within the technical field because hemodynamic monitoring is critical for patients suffering from a variety of different conditions (e.g., circulatory shock), especially in the ICU or other medical environments, as described in Cecconi et al., Consensus on Circulatory Shock and Hemodynamic Monitoring, Task Force of the European Society of Intensive Care Medicine. Intensive Care Med. 2014 December; 40(12):1795-815. doi: 10.1007/s00134-014-3525-z. In some cases, noninvasive hemodynamic monitoring tools have also been used in combination with other techniques (e.g., echocardiography) to noninvasively estimate cardiac filling and function. However, although echocardiography or other related techniques could be used in combination with noninvasive hemodynamic tools in a diagnostic manner, it is not practical to use these combinations of techniques for patient monitoring.

Therefore, there is a need in the technical field for improved non- or less-invasive patient-monitoring tools that are able to provide real-time hemodynamic parameters to medical staff. Further, these new tools need to be user-friendly to ensure adoption by medical staff. Therefore, the patient-monitoring tools should be intuitive and guide medical staff in using the tools in order to produce consistent, accurate, and verifiable results.

### SUMMARY

In some embodiments, there is provided a hemodynamic monitoring system comprising: an ultrasound system comprising a transesophageal ultrasound probe configured to obtain a view of a heart; and a computer system coupled to the ultrasound system, the computer system comprising a processor and a memory, the memory storing instructions that, when executed by the processor, cause the computer system to: receive a plurality of images of the heart from the ultrasound system obtained via the transesophageal ultrasound probe, identify, via a first machine learning system trained to identify a region of interest associated with a selected anatomical structure of the heart, the region of interest in the plurality of images, segment, via a second machine learning system trained to identify the selected anatomical structure using the identified region of interest, a predicted region corresponding to the selected anatomical from the plurality of images based on the identified region of interest, and calculate, based on the predicted region, a plurality of hemodynamic parameters associated with the heart, the plurality of hemodynamic parameters corresponding to a cardiac function and a cardiac filling.

In some embodiments, there is provided a hemodynamic monitoring system comprising: an ultrasound system comprising a transesophageal ultrasound probe configured to obtain a view of a heart; and a computer system coupled to the ultrasound system, the computer system comprising a processor and a memory, the memory storing instructions that, when executed by the processor, cause the computer system to: receive a plurality of images of the heart from the ultrasound system obtained via the transesophageal ultrasound probe, identify a selected anatomical structure associated with the heart from the received plurality of images, and determine, using a machine learning system trained to output an image quality parameter based on a visualization quality for the selected anatomical landmark in ultrasound images, the image quality parameter for the received plurality of images.

In some embodiments, the obtained view of the heart could include a transgastric short axis view or a mid-esophageal four chamber view.

### FIGURES

The accompanying drawings, which are incorporated in and form a part of the specification, illustrate the embodiments of the invention and together with the written description serve to explain the principles, characteristics, and features of the invention. In the drawings:

FIG. 1 illustrates a block diagram of a hemodynamic monitoring system, in accordance with an embodiment of the present disclosure.

FIG. 2 illustrates a diagram of the ultrasound system and ultrasound probe, in accordance with an embodiment of the present disclosure.

FIG. 3A illustrates a perspective view of the ultrasound probe, in accordance with an embodiment of the present disclosure.

FIG. 3B illustrates an exploded detail view of the interface between the transducer assembly and the actuator assembly of the ultrasound probe of FIG. 3A, in accordance with an embodiment of the present disclosure.

FIG. 3C illustrates a detail view of the interface portion of the actuator assembly of the ultrasound probe of FIG. 3A, in accordance with an embodiment of the present disclosure.

FIG. 3D illustrates a detail view of the interface portion of the transducer assembly of the ultrasound probe of FIG. 3A, in accordance with an embodiment of the present disclosure.

FIG. 3E illustrates a first detail view of the internal components of the transducer assembly of FIG. 3D with the lid removed, in accordance with an embodiment of the present disclosure.

FIG. 3F illustrates a second detail view of the internal components of the transducer assembly of FIG. 3D with certain components removed, in accordance with an embodiment of the present disclosure.

FIG. 3G illustrates a detail view of the electrical and mechanical interactions between the transducer assembly and the actuator assembly of the ultrasound probe of FIG. 3A, in accordance with an embodiment of the present disclosure.

FIG. 4 illustrates a block diagram of an image segmentation algorithm for identifying a structure in an ultrasound image, in accordance with an embodiment.

FIG. 5A illustrates an ultrasound image without augmentation, in accordance with an embodiment.

FIG. 5B illustrates an ultrasound image augmented with noise, in accordance with an embodiment.

FIG. 6 illustrates an ultrasound image with a predicted segmentation corresponding to a selected anatomical structure output by the algorithm illustrated in FIG. 4, in accordance with an embodiment.

FIG. 7 illustrates a diagram of an image quality assessment algorithm, in accordance with an embodiment.

FIG. 8A illustrates a series of ultrasound images having an image quality parameter output by the algorithm of FIG. 7 of less than 0.4, in accordance with an embodiment.

FIG. 8B illustrates a series of ultrasound images having an image quality parameter output by the algorithm of FIG. 7 of less than 0.6, in accordance with an embodiment.

FIG. 8C illustrates a series of ultrasound images having an image quality parameter output by the algorithm of FIG. 7 of less than 0.8, in accordance with an embodiment.

FIG. 8D illustrates a series of ultrasound images having an image quality parameter output by the algorithm of FIG. 7 of greater than 0.9, in accordance with an embodiment.

FIG. 9A illustrates a user interface provided by the hemodynamic monitoring system for a sepsis patient, in accordance with an embodiment.

FIG. 9B illustrates another embodiment of a user interface provided by the hemodynamic monitoring system for a sepsis patient, in accordance with an embodiment.

FIG. 10 illustrates a user interface provided by the hemodynamic monitoring system for a hypovolemic patient, in accordance with an embodiment.

FIG. 11 illustrates a user interface provided by the hemodynamic monitoring system for a cardiogenic shock patient, in accordance with an embodiment.

FIG. 12 illustrates a user interface provided by the hemodynamic monitoring system for setting hemodynamic targets, in accordance with an embodiment.

FIG. 13 illustrates a user interface provided by the hemodynamic monitoring system displaying values for the monitored hemodynamic parameters relative to user-defined targets, in accordance with an embodiment.

### DETAILED DESCRIPTION

The present disclosure is generally directed to hemodynamic monitoring systems and techniques for providing hemodynamic and image quality parameters from the ultrasound images in real-time, thereby allowing medical staff to non-invasively monitor patients and use the hemodynamic monitoring system equipment to produce good results. In some embodiments, the hemodynamic monitoring systems' algorithms (e.g., for image processing hemodynamic parameter generation, and image quality parameter generation) are adapted for use with ultrasound images obtained using a transesophageal ultrasound probe, which is described in detail below.

### Hemodynamic Monitoring System

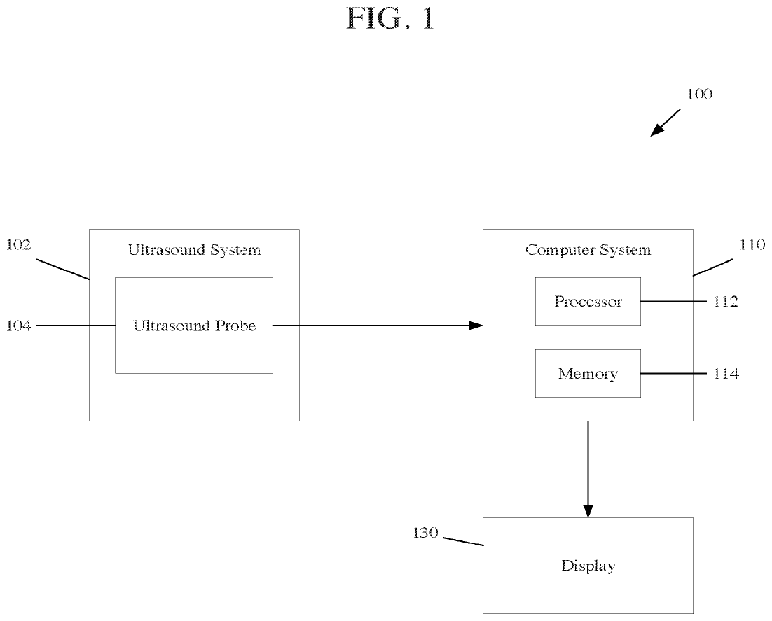

FIG. 1 illustrates a block diagram of a hemodynamic monitoring system 100 that includes an ultrasound system 102 and a computer system 110 communicatively coupled thereto. The ultrasound system 102 can include an ultrasound probe 104 that can be used to capture ultrasound images of a patient. In one embodiment, the ultrasound probe 104 can include a transesophageal probe suitable for use in, e.g., transesophageal echocardiography applications. When transesophageal echocardiography is used to obtain a transgastric short axis view of the left ventricle of the heart, the best place to position the transducer is in the fundus of the stomach, aimed up through the left ventricle of the patient's heart. Accordingly, transesophageal probes intended for such uses can be designed to facilitate placement of the transducer of the ultrasound probe 104 in the optimum position within the fundus, despite wide variations in the distance between the lower esophageal sphincter and the fundus among different subjects. In one embodiment, the ultrasound probe 104 can be configured to obtain a transgastric short axis view of a patient's heart. In another embodiment, the ultrasound prove 104 can be configured to obtain a mid-esophageal four chamber view or other such views of a patient's heart.

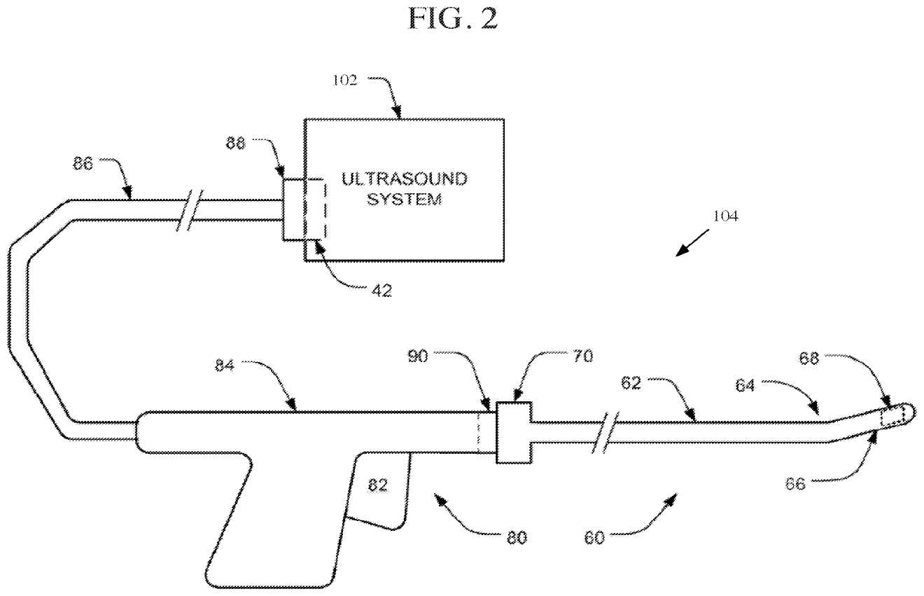

In one embodiment, illustrated in FIG. 2, the ultrasound probe 104 can include an actuator assembly 80 and a transducer assembly 60. The actuator assembly 80 includes a control handle 84 with an actuator 82. The handle 84 is connected to a connector 42 on the ultrasound system 102 via a cable 86 that terminates at a connector 88. The transducer assembly 60 has a flexible shaft 62 affixed to the end of a connector 70, and the distal end 66 of the probe contains the ultrasound transducer 68. To use the probe, the actuator assembly 80 and the transducer assembly 60 are connected together by mating the first connector 90 with the second connector 70. The distal end 66 is then manipulated into position in the esophagus. The transducer assembly 60 includes a bending mechanism that is actuatable by the actuator 82 when the actuator assembly 80 and the transducer assembly 60 are connected together.

When it is necessary to move the patient or it is desirable to remove components of the ultrasound probe 104 from the patient for the patient's comfort, the transducer assembly 60 can be disconnected from the actuator assembly 80 at the connectors 70, 90, so that the only parts that remain protruding from the patient will be the proximal end of the shaft 62 and the second connector 70. Since those portions are relatively small and light compared to the other components, the distal end of the probe can be left in the patient without causing the patient an undue amount of discomfort.

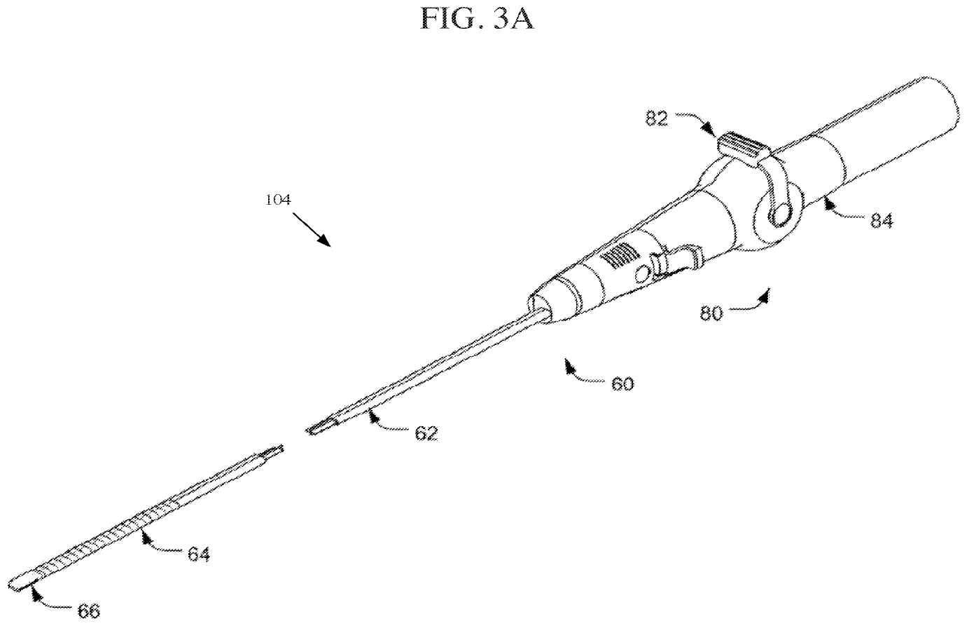

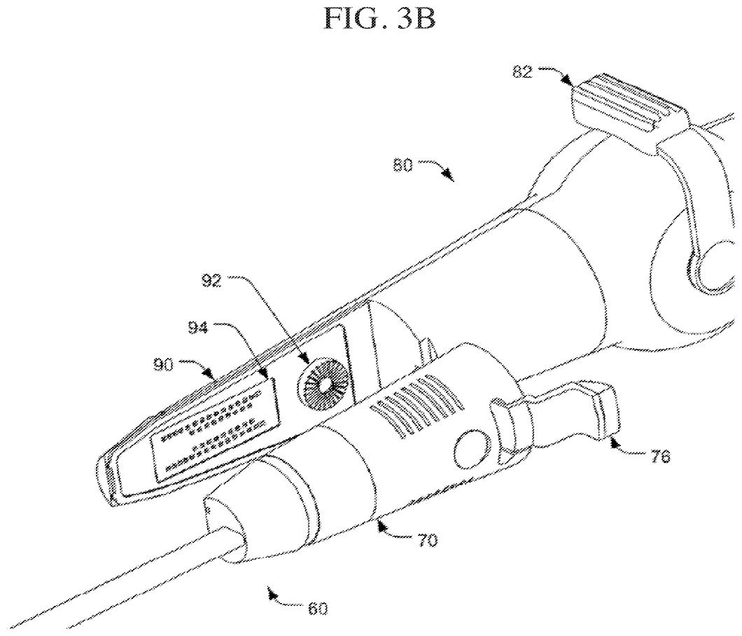

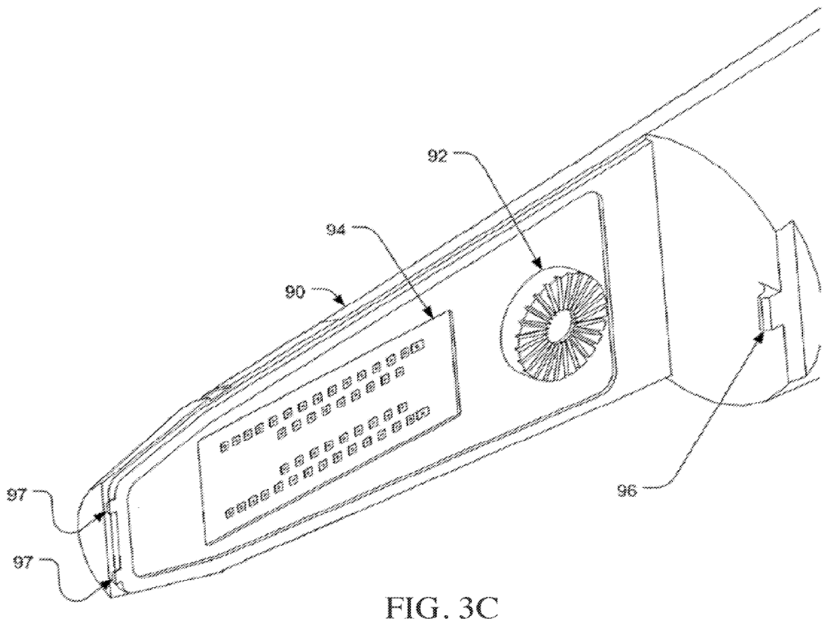

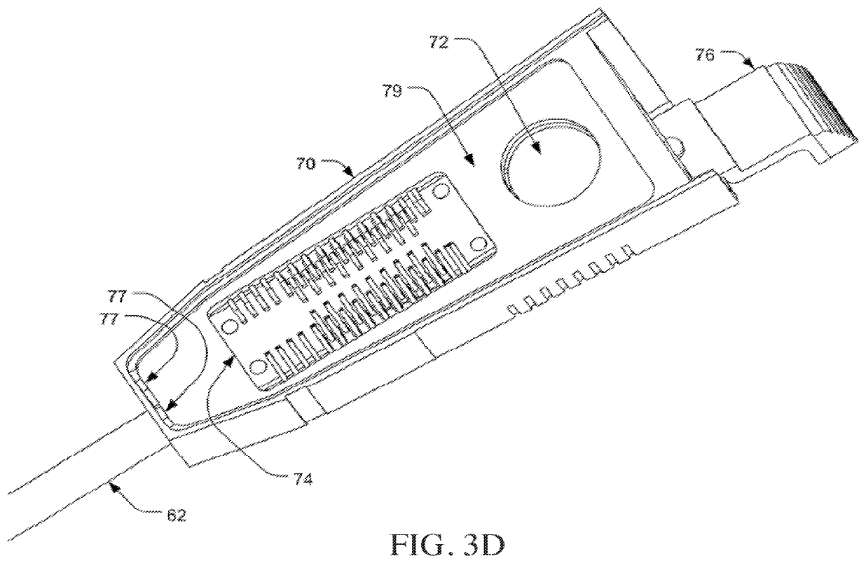

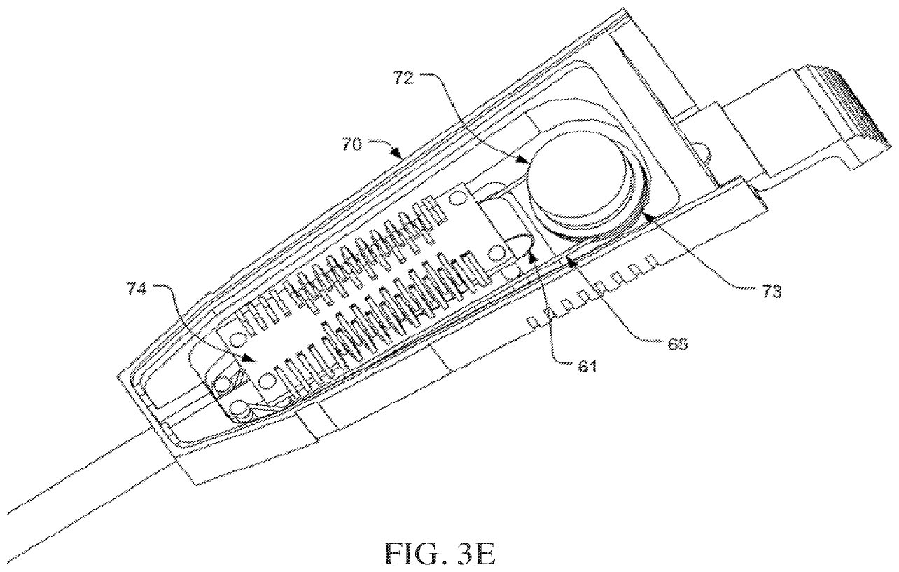

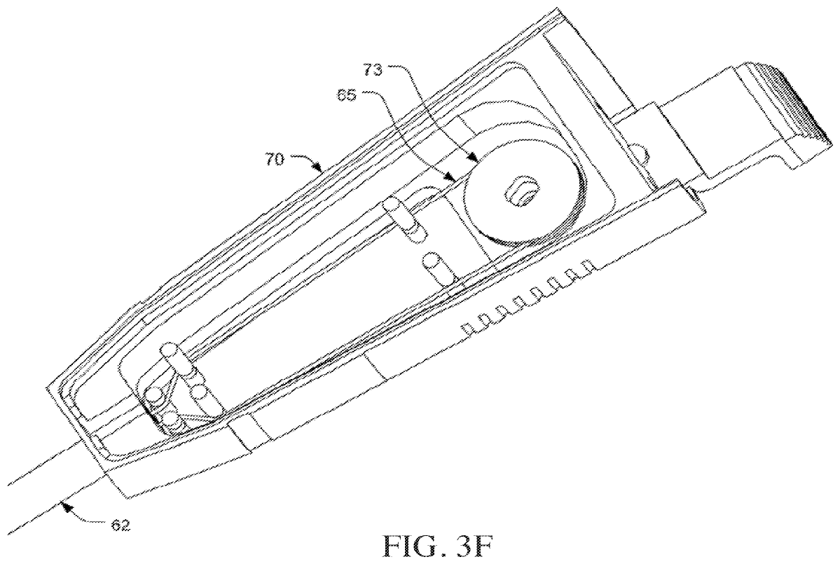

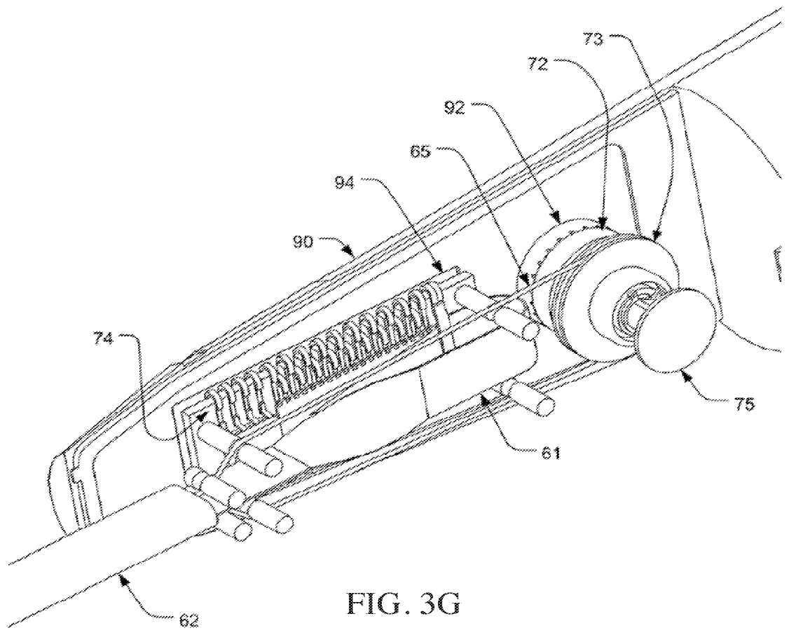

FIGS. 3A-3G depict one embodiment of the ultrasound probe 104 with distal and proximal portions that can be reversibly disconnected from each other. In particular, the transducer assembly 60 can be mounted to the actuator assembly 80. The transducer assembly 60 includes a flexible shaft 62 (shown with a break to denote its long length) that has a bending section 64. In various embodiments, the shaft 62 can be less than 6 mm in diameter and approximately 1 m in length for an adult version of the device. Those dimensions may be scaled down appropriately for pediatric and neonatal patients. The distal end 66 of the transducer assembly 60 houses the ultrasound transducer which is preferably transversely oriented with respect to the proximal distal axis. In alternative embodiments, other transducer configurations may be used in place of the transversely oriented transducer (e.g., a two-dimensional ultrasound transducer or a rotating multi-plane transducer). The actuator assembly 80 includes a handle 84 with a user-operated actuator 82 mounted on the handle 84. A cable 86 with a connector 88 at its proximal end (both shown in FIG. 2) extends from the proximal end of the handle 84. This connector 88 mates with a corresponding connector 42 on the ultrasound system 102 (all shown in FIG. 2).

FIG. 3B illustrates an exploded detail view of the interface between the actuator assembly 80 and the transducer assembly 60. The actuator assembly 80 includes a first connector 90 that interfaces with the transducer assembly 60, and the transducer assembly 60 includes a second connector 70 that interfaces with the actuator assembly 80. The first connector 90 includes a first electrical interface 94, which is used to make electrical connect with a mating connector (not shown) on the second connector 70. In the illustrated embodiment, the first electrical interface 94 comprises a series of conductive pads, which are preferably gold plated. The pads may be flat or raised. Preferably, the first connector 90 is constructed to be watertight so that the first connector 90 can be immersed in a liquid sterilant (e.g., Cidex glutaraldehyde or peroxide sterilants), and using simple, stationary pads helps achieve the desired watertightness, which facilitates re-use of the actuator assembly 80 for multiple patients. When the second connector 70 is mated to the first connector 90, corresponding contacts on the second connector 70 line up with the contacts of the first electrical interface 94 so that electrical signals can pass between the actuator assembly 80 and the transducer assembly 60.

The ultrasound system 102 communicates with the ultrasound transducer 68 (both shown in FIG. 2) by sending and receiving appropriate signals into the actuator assembly 80 via the connector 42, the connector 88, and the cable 86 (all shown in FIG. 2). The signals that travel through the cable 86 are routed to the first electrical interface 94 on the first connector 90 e.g., by running appropriately shielded wires from the distal end of the cable 86 directly to the first electrical interface 94. Optionally, appropriate intervening circuitry (e.g., amplifiers and signal conditioners) may be interposed between the first electrical interface 94 and the cable 86. The remainder of the path to the transducer is described below in connection with the transducer assembly 60.

The first connector 90 also includes an output actuator 92 that is designed to mate with a corresponding member on the second connector 70 when the second connector 70 is connected to the first connector 90. The output actuator 92 is linked to the user-operated actuator 82 by an appropriate mechanism such that the output actuator moves in response to user actuation of the user-operated actuator 82. The link between the user-operated actuator 82 and the output actuator 92 may be implemented using any of a variety of conventional techniques, including but not limited to gears, pull wires, servo motors, stepper motors, hydraulics, as well as numerous other techniques that will be apparent to persons skilled in the relevant arts. The output actuator 92 and the user-operated actuator 82 are preferably also made using a watertight construction (e.g., using 0 rings or other sealing techniques) to facilitate liquid sterilization of the actuator assembly 80.

FIG. 3C shows a detail view of the first connector 90. As explained above, the output actuator 92 rotates in response to actuations of the user-operated actuator 82. The surface of the output actuator 92 is preferably made of a material that will have a high coefficient of friction when it is pressed against a corresponding member in the second connector 70. Examples of suitable materials for the output actuator include rubber, polyethylene, polystyrene, vinyl, etc. Optionally, a plurality of radial grooves may be cut into the surface of the output actuator 92 to help the output actuator 92 better “grab” the corresponding surface on the second connector 70.

As best seen in this view, the first connector 90 includes a number of mounting members for latching the first connector 90 onto the second connector 70. Although the illustrated embodiment depicts mounting members in the form of a pair of small tabs 97 at the distal end and a larger tab 96, persons skilled in relevant arts will recognize that any of a wide variety of conventional latching mechanisms may be used.

FIG. 3D shows a front view of the second connector 70. The second connector 70 is configured to mate with the first connector 90. To do this, the second connector 70 contains a second electrical interface 74 that lines up the first electrical interface 94 of the first connector 90. In the illustrated embodiment, the second electrical interface 74 is made using a plurality of spring loaded fingers positioned so that, when the second connector 70 is connected to the first connector 90, the fingers of the second electrical interface 74 will line up with the pads of the first electrical interface 94 (shown in FIGS. 3B and 3C). The second connector 70 also contains a control actuator 72 that lines up the output actuator 92 of the first connector 90, so that the output actuator 92 can drive the control actuator 72. In the illustrated embodiment, the control actuator 72 is a rotating wheel that is designed to be driven by rotation of the output actuator 92. Of course, a wide variety of alternative arrangements for actuating alternative control actuators will be readily apparent to persons skilled in the relevant arts. Note that when the transducer assembly 60 is disposable and will be discarded after each use, it is not necessary to make the second connector 70 watertight.

To connect the first and second connectors, the second connector 70 is attached to the first connector 90 by aligning the notches 77 of the second connector 70 with tabs 97 of the first connector 90, then squeezing the proximal end of second connector 70 towards the first connector 90. The latching arm 76 on the second connector 70 is designed to snap into position on the first connector by interacting with tab 96 (shown in FIG. 3C). When the second connector 70 is attached to the first connector 90 in this manner, the second electrical interface 74 of the second connector 70 makes electrical contact with the first electrical interface 94 of the first connector 90, so that electrical signals can travel back and forth between the first electrical interface 94 and the second electrical interface 74. In addition, the control actuator 72 makes mechanical connect with the output actuator 92 of the first connector 90, so that when the output actuator 92 is rotated in response to operation of the user operated actuator 82 (shown in FIG. 3B) the control actuator 72 will be driven by the output actuator 92 and followed by the rotation of the output actuator 92. A lid 79 protects the internal components of the second connector 70 from damage, and has cutouts to provide access to the second electrical interface 74 and the control actuator 72. Note that while the first and second electrical interfaces 94, 74 are depicted using pads and fingers designed to contact the pads, numerous alternative electrical interfaces (e.g., pins and mating sockets) may be substituted therefore, as will be appreciated by persons skilled in the technical field.

FIG. 3E is another view of the second connector 70 shown in FIG. 3D, with the lid 79 removed. This view reveals that the rotating control actuator 72 is attached to a pulley 73 that causes the pull wires 65 to move when the control actuator 72 is rotated. This view also shows a portion of the wiring 61 (e.g., a ribbon cable), which is the wiring that connects the second electrical interface 74 to the transducer 68 (shown in FIG. 2) at the distal end 66 of the transducer assembly 60. Preferably, a ground plane is provided on both sides of the ribbon cable. In less preferred embodiments one or both of those ground planes may be omitted, or wiring configurations other than ribbon cable may be used. Optionally, appropriate intervening circuitry (e.g., amplifiers and signal conditioners) may be interposed between the second electrical interface 74 and the transducer 68.

FIG. 3F shows yet another view of the second connector 70 of FIGS. 3D and 3E, but with the lid 79, the second electrical interface 74, the wiring 61, the control actuator 72, and the pulley's axle all removed to show the lower components of the second connector 70. This view more clearly shows how the pulley 73 moves the pull wires 65, which extend out distally through the shaft 62. When the pull wires 65 move (in response to rotation of the pulley), the pull wires operate the bending section 64 (shown in FIG. 3A) in any conventional manner. Since the pull wires 65 cause the bending section 64 to bend, and the pull wires 65 are moved by rotation of the pulley 73, and rotation of the pulley 73 occurs in response to rotation of the control actuator 72 (shown in FIGS. 3D and 3E), the net result is that rotation of the control actuator 72 causes the bending section 64 to bend.

FIG. 3G shows the electrical and mechanical interactions between the first connector 90 and the second connector 70 when those connectors are mated together. This view depicts how the mated set of connectors 90, 70 would look if the outside housing of the second connector 70 were invisible. The second electrical interface 74 is lined up with and urged against the first electrical interface 94, and the control actuator 72 on the second connector 70 is lined up with and urged against the output actuator 92 on the first connector 90. A pulley mount 75 permits the pulley 73 to rotate and urges the control actuator 72 against the output actuator 92 when the first connector 90 and second connector 70 are mated. The wiring 61 (e.g., a ribbon cable) that connects the second electrical interface 74 to the transducer 68 (shown in FIG. 2) at the distal end 66 of the transducer assembly 60 is also more clearly visible in this view.

When the second connector 70 is mated with the first connector 90, actuation of the user operated actuator 82 (shown in FIGS. 3A and 3B) will cause the output actuator 92 to rotate. Since the control actuator 72 is being urged up against the output actuator 92, the control actuator 72 will follow the rotation of the output actuator 92. Rotation of the control actuator 72 turns the pulley 73 which operates the pull wires 65 that extend distally through the flexible shaft 62, and cause a bending mechanism (not shown) located in the bending section (shown in FIG. 3) to bend. Note that while rotating pads are depicted for the output actuator 92 and the control actuator 72 pads, numerous alternative mechanical interfaces (e.g., gears, a hexagonal shaft and a mating socket, etc.) may be substituted therefore, as will be appreciated by persons skilled in the technical field.

In addition, when the second connector 70 is mated with the first connector 90, the second electrical interface 74 makes contact with the first electrical interface 94. Since the first electrical interface 94 communicates with the ultrasound system 102 via cable 86 and connectors 88, 42 (all shown in FIG. 2). Since the wiring 61 connects the second electrical interface 74 to the transducer 68 at the distal end 66 of the transducer assembly 60 (shown in FIGS. 2 and 3A) this arrangement permits the ultrasound system 102 to interface with the transducer 68. Optionally, additional signals may be passed to and from the transducer assembly 60 via the first and second connectors 90, 70, e.g., to operate a thermistor located in the distal end of the transducer assembly 60 or to interface with a non-volatile memory device located in the transducer assembly 60 (used, e.g., to store data relating to the transducer assembly 60).

In one particular embodiment, the ultrasound probe 104 can include a connectorized ultrasound probe comprising a compact first section having a distal end that is configured for insertion into a patient's body, with an ultrasound transducer located in the distal end, and a second section configured to provide an electrical interface between the first section and an ultrasound system. The first section can be attachable and detachable from the second section using at least one set of connectors. The second section includes at least one user-operated actuator and the first and second sections are configured so that, when the first section is attached to the second section, actuation of the user-operated actuator causes the first section to bend. Further, the first and second sections can be configured so that, when the first section is attached to the second section, (a) the ultrasound system can drive the ultrasound transducer by sending drive signals into the first section via the second section and (b) the ultrasound transducer in the first section can send return signals to the ultrasound system via the second section. Finally, the transducer can be transversely oriented with respect to a proximal-distal direction axis of the first section, wherein the first section is configured so that when the first section is inserted into the patient's esophagus with the ultrasound transducer positioned in the patient's stomach fundus, the portion of the first section that remains outside of the patient's body has a length of 70 cm or less and a mass of 250 g or less, and wherein the first section is sealed to prevent the entry of liquids.

Various additional details regarding embodiments of the ultrasound probe 104 can be found in U.S. Pat. No. 8,070,685, titled CONNECTORIZED PROBE FOR TRANSESOPHAGEAL ECHOCARDIOGRAPHY, filed Apr. 12, 2006 and U.S. Pat. No. 8,579,822, titled TRANSESOPHAGEAL ULTRASOUND PROBE WITH AN ADAPTIVE BENDING SECTION, filed Mar. 5, 2007, each of which is hereby incorporated by reference herein in its entirety.

In one embodiment, the transducer 68 can be a phased array transducer made of a stack of N piezo elements, an acoustic backing, and a matching layer, as is generally known to those skilled in the technical field. As understood by persons skilled in the technical field, the elements of phased array transducers can be driven individually and independently, without generating excessive vibration in nearby elements due to acoustic or electrical coupling. In addition, the performance of each element can be as uniform as possible to help form a more homogeneous beam. Optionally, apodization may be incorporated into the transducer (i.e., tapering the power driving transducer elements from a maximum at the middle to a minimum near the ends in the azimuthal direction, and similarly tapering the receive gain).

The ultrasound system 102 can be configured to provide signals to drive the transducer 68 through the probe 104 via appropriate wiring and any intermediate circuitry. Further, the ultrasound system 102 can be configured to receive return signals from the transducer 68 through the probe 104. The return signals can ultimately be processed into images by the ultrasound system 102 and/or the computer system 110. The images can then be displayed on a display 130 coupled to the computer system 110, for example.

Returning to FIG. 1, the computer system 110 can be configured to receive ultrasound signals and/or images captured by the ultrasound system 102 (in particular, using the various embodiments of the ultrasound probe 104 described above) and perform various processing techniques and/or execute various algorithms in order to identify structures within the images, calculate or generate data from the images, display the images to users (e.g., medical staff members), and so on. In various embodiments, the computer system 110 can be configured to receive individual ultrasound images and/or a video feed of ultrasound images. In one embodiment, the computer system 110 can be communicatively coupled to a display 130, which could include a display screen present within a hospital room or an operating room, for example.

Various embodiments of the processing techniques are described below. The algorithms, machine learning system, and/or techniques described below that are executed by the computer system 110 can be embodied as software, hardware, firmware, or combinations thereof. In one embodiment, the algorithms, machine learning system, and/or techniques executed by the computer system 110 can be embodied as instructions stored in a memory 114 of the computer system 110 that, when executed by a processor 112 coupled to the memory 114, cause the computer system 110 to perform the described steps of the processes.

Determining Hemodynamic Parameters from Ultrasound Images

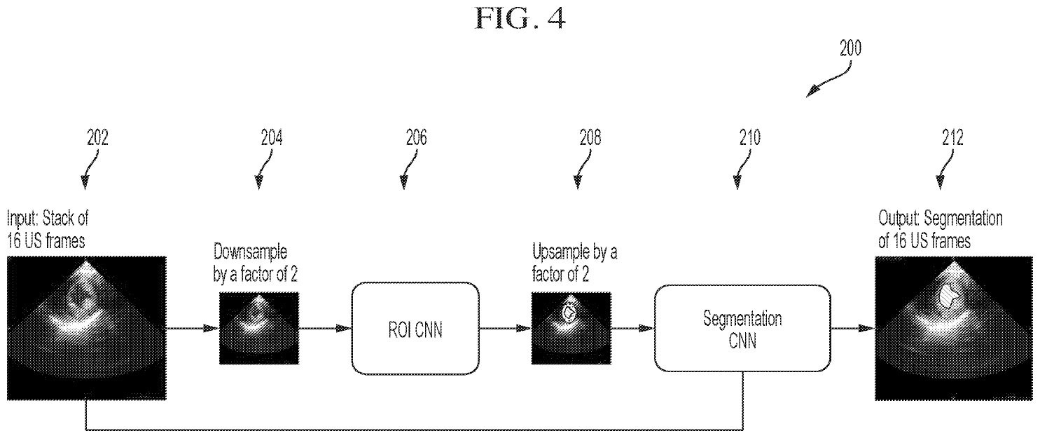

In some embodiments, the hemodynamic monitoring system 100 can be configured to identify a selected anatomical structure (e.g., a left ventricle of a patient's heart) in ultrasound images obtained via the ultrasound system 102. For example, the computer system 110 can be configured to execute an image segmentation machine learning algorithm 200, such as is shown in FIG. 4. As noted above, the image segmentation machine learning algorithm 200 can be embodied as instructions stored in the memory 114 of the computer system 110 that can be executed by the processor 112. In one embodiment, the image segmentation machine learning algorithm 200 can include a first machine learning system 206 (e.g., a convolutional neural network), which has been trained to identify a region of interest (ROI) corresponding to the selected anatomical structure in one or multiple ultrasound images, and a second machine learning system 210 (e.g., a convolutional neural network), which has been trained to segment the portion of the ultrasound image within the identified ROI that is predicted to correspond to the selected anatomical structure. In other words, the first machine learning system 206 can perform a “coarse” identification of the area in the images corresponding to the anatomical structure and the second machine learning system 210 can perform a further “fine” identification. In embodiments where one or both of the machine learning systems 206, 210 are convolutional neural networks, the machine learning system(s) 206, 210 can be based on, for example, the U-Net architecture, which is described in “U-net: Convolutional networks for biomedical image segmentation” by Ronneberger et al., 2015, October, International Conference on Medical Image Computing and Computer-Assisted Intervention (pp. 234-241), which is hereby incorporated by reference herein in its entirety. After segmenting the selected anatomical structure from the received ultrasound images, the computer system 110 can calculate or quantify one or more hemodynamic parameters associated with the segmented image of the structure.

In various embodiments, the machine learning systems 206, 210 can be trained using supervised or unsupervised learning techniques. For example, the machine learning systems 206, 210 can be trained via supervised learning techniques by providing the machine learning systems 206, 210 with ultrasound images containing the particular anatomical structure that have been manually annotated by medical professionals. The annotated ultrasound images can be divided into both training and validation data sets, as is generally known in the technical field. Further, the machine learning systems 206, 210 can be trained and validated with ultrasound images obtained at different depths of ultrasound penetration because the ultrasound penetration depth can affect the shape, location, and the size of anatomical structures in ultrasound images. Therefore, providing images of varying ultrasound training depths assists the machine learning systems 206, 210 in being robust to size and anatomical variation during operation.

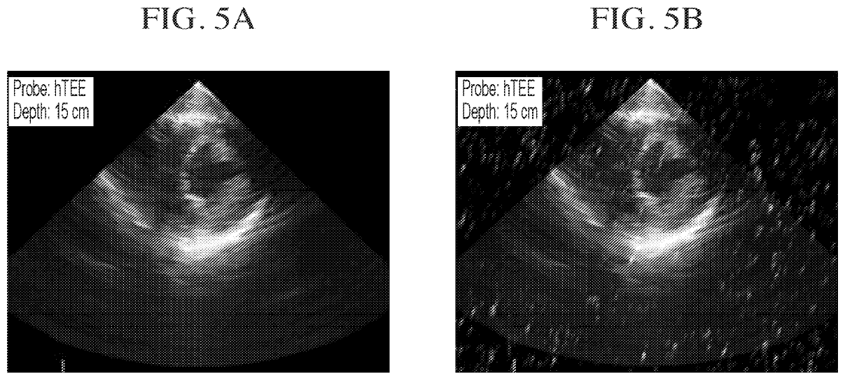

Further, due to variability in operator skill, challenges in placing the ultrasound probe 104, anatomical variation (e.g., differences in the size and/or shape of the anatomical structure between patients), ultrasound penetration depth, image quality variability, and other factors, ultrasound images of anatomical structures can in turn be highly variable. The image segmentation machine learning algorithm 200. Therefore, it would be desirable for the image segmentation machine learning algorithm 200 to be robust to these various factors. Accordingly, the machine learning systems 206, 210 can be trained and validated with various types of augmentations applied to the data. For example, the training and validation data sets can include images that can be augmented with images that have been altered using a variety of different techniques. For example, an augmented set of images can be created by zooming in on or out from the anatomical structure; adjusting the edges of the annotations for the anatomical structure (e.g., shifting the edges by a few pixels) to make the machine learning systems 206, 210 focus less on the edges of the anatomical structure and be robust to annotator variability; zooming in on or out from the annotations; blurring, sharpening, an/or adjusting the intensity of the images to make the machine learning systems 206, 210 more robust to noise artifacts and imaging loop qualities; rotating the images by an amount (e.g., 10 degrees) to make the machine learning systems 206, 210 robust to variability in the locations and angles of the ultrasound probes 104 when capturing images; and/or flipping the images to force the machine learning systems 206, 210 to look only at the region of the anatomical structure. One additional illustrative data augmentation technique is shown in FIGS. 5A and 5B. In this technique, various artifacts or noise (i.e., speckles) are added to the ultrasound images used to train and validate the machine learning systems 206, 210 to make the image segmentation machine learning algorithm 200 more robust to random white artifacts that commonly appear in ultrasound images.

Some data augmentation techniques can also be used that are specific to the imaging constraints particular to the specific anatomical structure. For example, in embodiments where the anatomical structure is the left ventricle of a heart, in some cases the lateral ventricle walls may be obscured or not visible. Accordingly, in one embodiment, an additional data augmentation step was used in the training and validation of the machine learning systems 206, 210 to account for this factor. In this embodiment, the training and validation data was augmented by introducing images where the lateral walls of the ventricle were obscured. The loss function used when training the machine learning system 206, 210 can also be tailored to such situations. In one embodiment, a loss function was utilized in training the machine learning systems 206, 210 that forced them to output a rough ellipse fit of the left ventricle area, in addition to the existing segmentation output. This additional loss served as a geometric constraint for the machine learning systems 206, 210, forcing the systems 206, 210 to include the lateral areas of the ventricle in its output segmentation even when an explicit border is not visible in the ultrasound images. It was empirically determined that the lateral wall dropout data augmentation technique and the ellipsoidal loss function provided similar results, ensuring that the machine learning systems 206, 210 fill out the expected boundaries of the left ventricle, even in situations where it cannot be seen in the frame. These techniques also resulted in more rounded and smooth predictions.

Returning to FIG. 4, there is shown a flowchart demonstrating the execution of the image segmentation machine learning algorithm 200 as executed by the computer system 110. Initially, the computer system 110 generates and/or receives 202 one or more ultrasound image frames (e.g., a stack of 16 frames) from the ultrasound system 102 and downsamples 204 the received image frames by a factor (e.g., a factor of two). The downsampled ultrasound images are input to the first machine learning system 206, which has been trained to output a ROI identified across the downsampled images. The first machine learning system 206 has been trained to output a ROI that is associated with the particular anatomical structure (e.g., a left ventricle). The images with the identified ROI are then upsampled 208 by a factor (e.g., a factor of two), which can be the same or different than the downsampling factor. The resulting upsampled images are input to the second machine learning system 210 that has been trained to segment or identify the anatomical structure (e.g., a left ventricle) across the images. The second machine learning system 210 outputs 212 segmented images that visually indicated the predicted region corresponding to the anatomical structure.

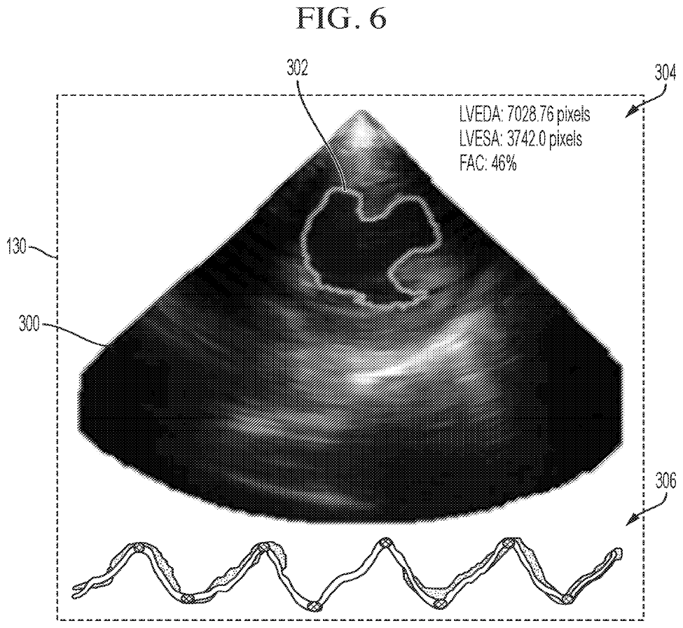

The segmented images can be used in a variety of different ways. In one embodiment, the computer system 110 can display the segmented images to users, such as via the display 130. The segmented images can be displayed intraprocedurally or in real-time, for example. FIG. 6 illustrates an example of a segmented ultrasound image 300 displayed via the display 130, including the predicted region 302 in the image 300 corresponding to the anatomical structure (which, in this particular case, is a left ventricle).

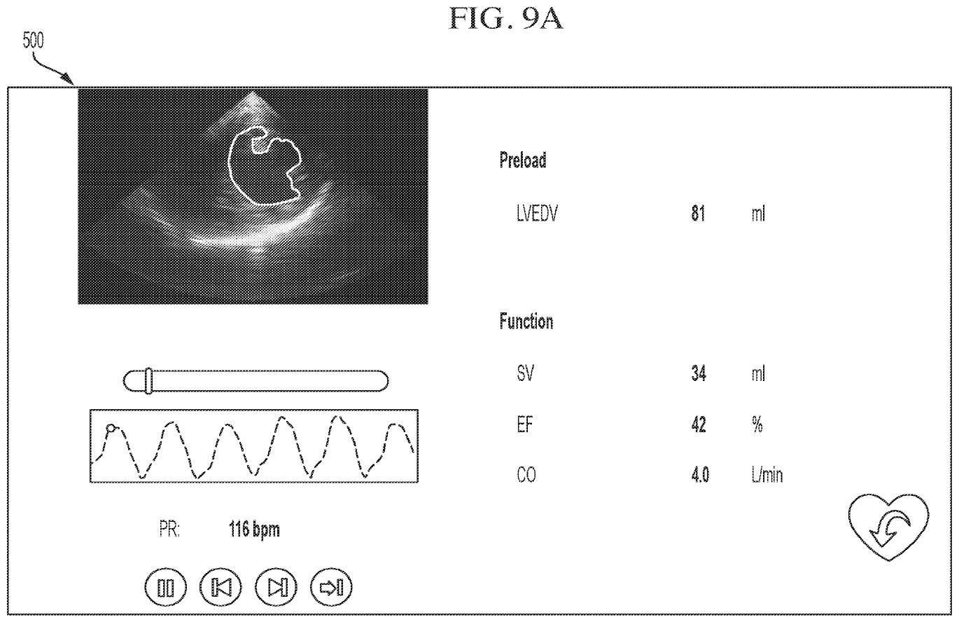

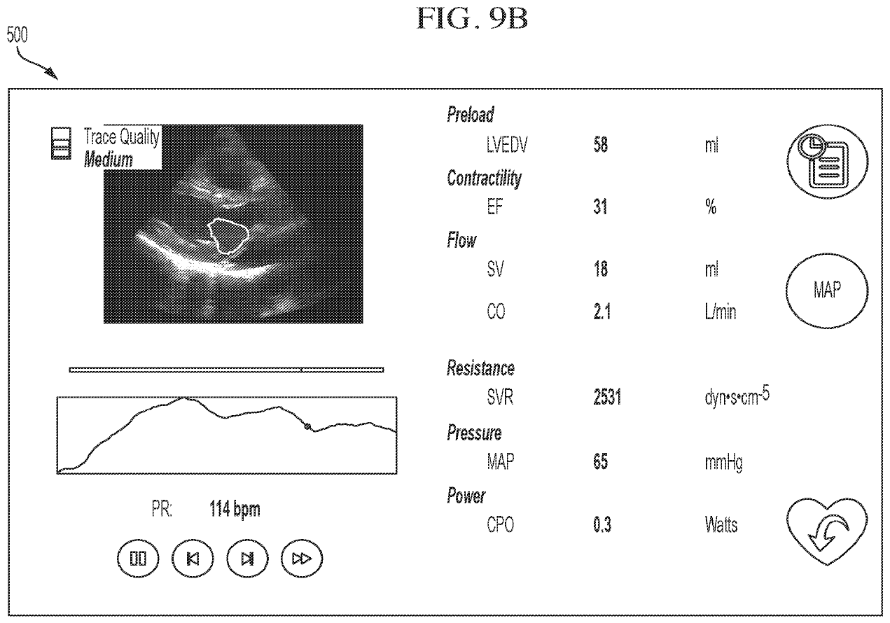

In another embodiment, the computer system 110 can calculate one or more hemodynamic parameters 304 corresponding to the anatomical structure from the predicted region 302. For example, FIG. 6 illustrates an example where the computer system 110 has calculated the left ventricular end-diastolic area (LVEDA), left ventricular end-systolic area (LVESA), and the fractional area change (FAC), wherein FAC=(LVEDA−LVESA)/LVEDA. FAC is a measurement that provides an estimate of the global right ventricular systolic function by calculating the % change of area within the right ventricle between diastole and systole. A normal value for the FAC is 50% or higher. As can be seen in FIG. 6, the computer system 110 can display the hemodynamic parameters 304 to users, such as via the display 130. The computer system 110 can also display the hemodynamic parameters 304 in a variety of different ways. For example, the computer system 110 can display the numerical values of the calculated hemodynamic parameters 304 or alternative visualizations, such as a line graph 306 of the change in the patient's FAC over time. In other embodiments, a variety of other hemodynamic parameters can be calculated or generated by the computer system 110 from the projected region 302 of the anatomical structure output by the image segmentation machine learning algorithm 200, such as FAC, heart rate, stroke volume (SV), cardiac output (CO), left ventricular end diastolic volume (LVEDV), ejection fraction (EF), systemic vascular resistance (SVR), cardiac power output (CPO), and so on. These and other hemodynamic parameters can be calculated either directly from the processed ultrasound images or as secondary parameters calculated from the primary parameters calculated from the processed ultrasound images. In some embodiments, the hemodynamic parameters could be calculated based on additional data and/or input (e.g., provided by users or retrieved from patient medical record databases). For example, SVR and CPO, as generally shown in FIG. 9B, could be calculated based at least in part on the patient's mean arterial pressure (MAP), which could be input to the hemodynamic monitoring system 100 by users or received from other monitoring devices coupled to the hemodynamic monitoring system 100.

### Ultrasound Image Acquisition Assistance

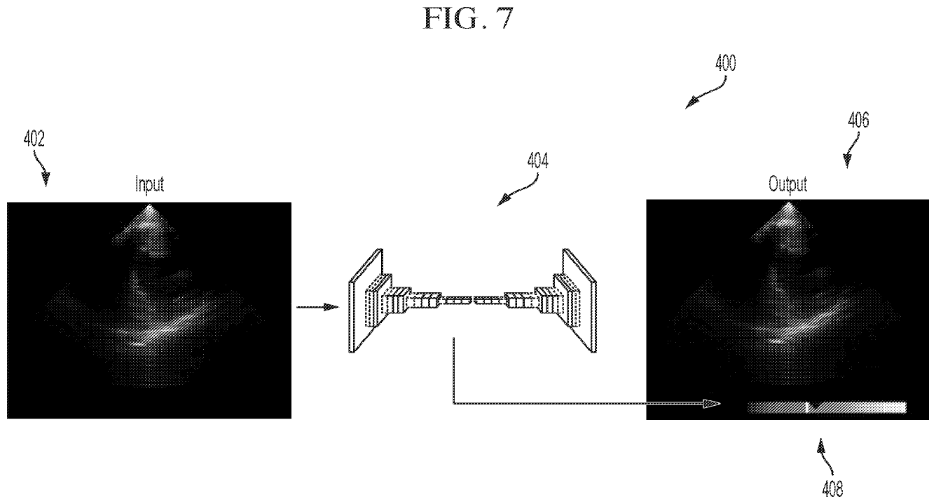

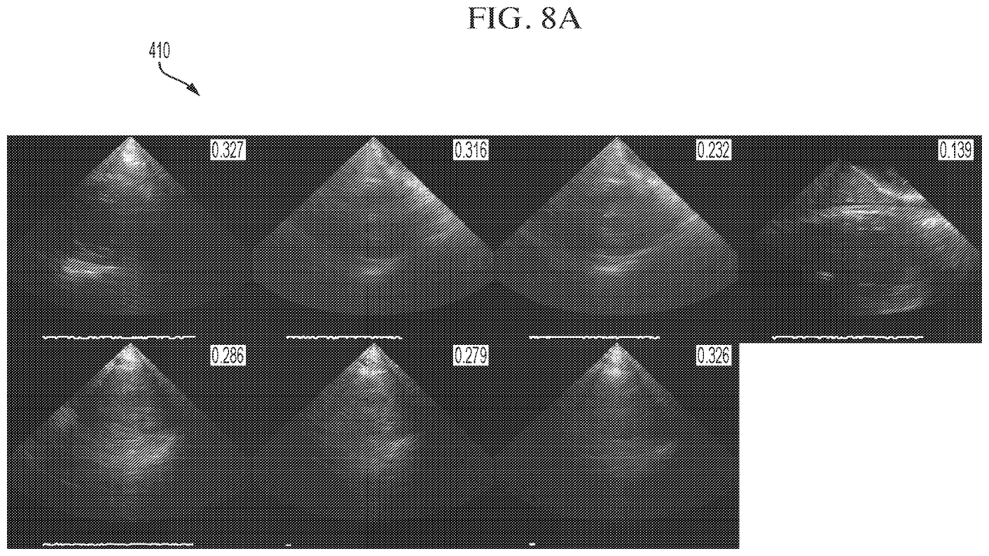

In some embodiments, the hemodynamic monitoring system 100 can be configured to assist users in obtaining high quality visualizations of the selected anatomical structure. For example, the computer system 110 can be configured to execute an image quality machine learning algorithm 400 that is configured to indicate whether a particular ultrasound image or series of images is of appropriate quality for the selected anatomical structure, as shown in FIG. 7. In one embodiment, the image quality machine learning algorithm 400 can include a machine learning system 404 that has been trained to output a quality score (e.g., which can be displayed to users) indicating the quality of the visualization of the selected anatomical structure in the ultrasound image(s). As noted above, the image quality machine learning algorithm 400 can be embodied as instructions stored in the memory 114 of the computer system 110 that can be executed by the processor 112. In one embodiment, the machine learning system 404 could include a convolutional neural network based on the U-Net architecture described above, for example.

In various embodiments, the machine learning system 404 can be trained using supervised or unsupervised learning techniques. For example, the machine learning system 404 can be trained via supervised learning techniques by providing the machine learning system 404 with ultrasound images containing the particular anatomical structure that have been manually annotated by medical professionals. Accordingly, the machine learning system 404 can be trained to output an image quality parameter based on the training data provided thereto. In one embodiment, the image quality metric could include a dice score, such as is described in “Optimizing the Dice score and Jaccard index for medical image segmentation: Theory and practice” by Bertels et al., 2019, October, International Conference on Medical Image Computing and Computer-Assisted Intervention (pp. 92-100), which is hereby incorporated by reference herein in its entirety. In one embodiment, training the machine learning system 404 can consist of providing the machine learning system 404 with sets of non-augmented and augmented ultrasound images of patients with the selected anatomical structure visible in the images. The augmented ultrasound image sets can be examined visually to verify that they look like real cases. Augmented data can be generated so that the machine learning system 404 can be trained on a full spectrum of good and poor quality data. In various embodiments, the ultrasound image data can be augmented using any of the techniques described above. Further, the machine learning system 404 can go through multiple rounds of training. In one application, round of training can utilize a subset (e.g., 80%) of the data for training and a second subset (e.g., 20%) for validation, as is generally known in the art. Further, no ultrasound image sets were included in both the training and validation datasets in each training session to ensure that the machine learning system 404 was not being validated on images from patients that it had already been trained on during that session. After completing the training, the image quality parameter predictions output by the machine learning system 404 were evaluated against user-determined calculations for the image quality parameter to evaluate the performance of the machine learning system 404. This process was repeated with additional training sessions until the performance of the machine learning system 404 was deemed to be adequate.

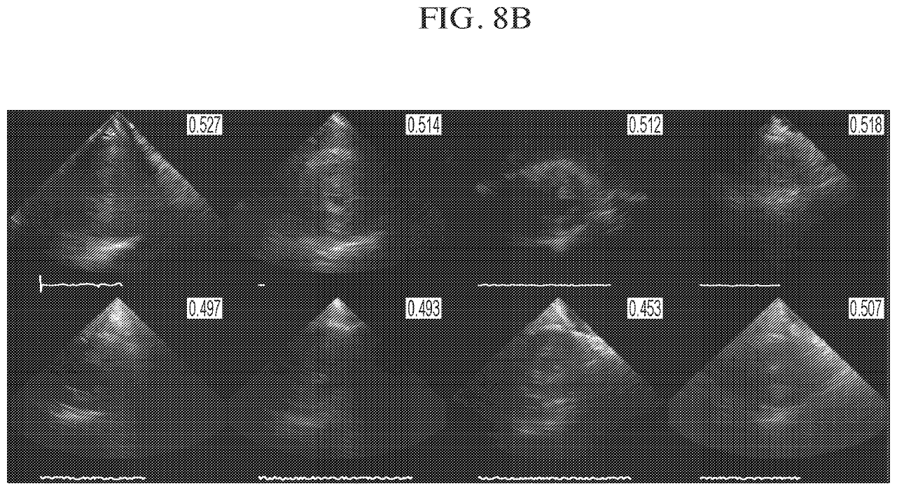

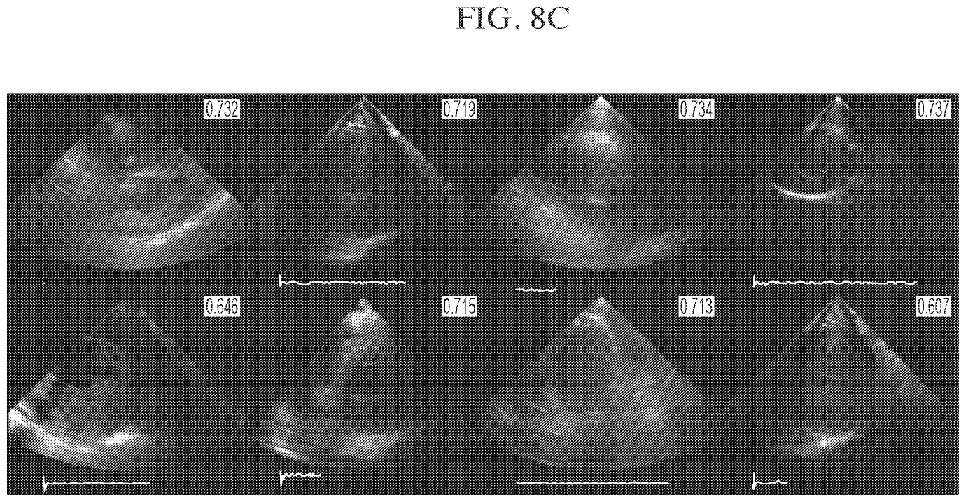

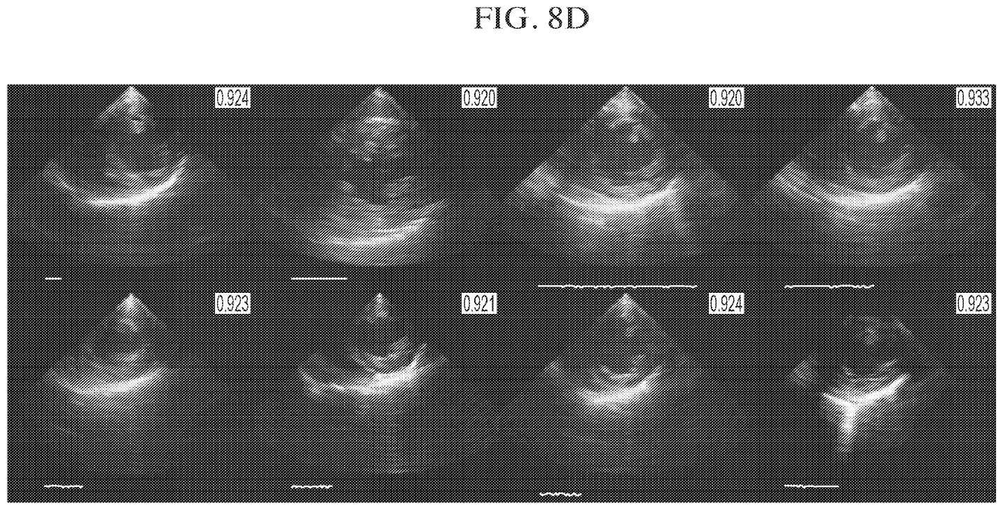

Accordingly, the computer system 110 generates and/or receives 402 one or more ultrasound image frames from the ultrasound system 102. The received ultrasound images are input to the machine learning system 404 (which has been trained as described above), which accordingly outputs 406 an image quality parameter based on the quality of the visualization of the selected anatomical structure (e.g., the left ventricle). In one embodiment, the computer system 110 can further display the calculated image quality parameter to users, such as via the display 130. The computer system 110 can also display the hemodynamic parameters 304 in a variety of different ways. For example, the computer system 110 can display the numerical values 410 of the calculated image quality parameter (as shown in FIGS. 8A-8D), graphical element (e.g., a heat bar 408 as show in FIG. 7) indicating calculated image quality parameter, and other types of visualizations. Further, in some embodiments the computer system 110 can be configured to calculate and display both an image quality parameter for a particular individual ultrasound image frame and a running weighted-average image quality parameter for a number of ultrasound images frames (e.g., for a loop of ultrasound images). FIG. 8A demonstrates ultrasound images having a quality score calculated by the image quality machine learning algorithm 400 that is less than 0.4, FIG. 8B demonstrates ultrasound images having a quality score less than 0.6, FIG. 8C demonstrates ultrasound images having a quality score less than 0.8, and FIG. 8D demonstrates ultrasound images having a quality score greater than 0.9.

The image segmentation machine learning algorithm 200 and the image quality machine learning algorithm 400 described above can be executed by the computer system 110 either individually or in combination with each other. In other words, the computer system 110 could be programmed or otherwise configured to simultaneously calculate and display the hemodynamic parameters and the image quality parameters to users (e.g., via the display 130). This could be beneficial because it would allow the medical staff to determine whether the ultrasound probe 104 or other components of the hemodynamic monitoring system 102 should be adjusted to provide higher quality images, which could in turn affect the calculation of the hemodynamic parameters. Accordingly, the image segmentation machine learning algorithm 200 and the image quality machine learning algorithm 400 can function synergistically in combination with each other in operation of the hemodynamic monitoring system 100. However, as noted above, the algorithms 200, 400 could also be utilized in their individual capacities. Various other signal processing or other imaging techniques are can be utilized by the hemodynamic monitoring system 100 in combination with or in lieu of the techniques described above, including the techniques disclosed in U.S. Pat. No. 7,998,073, titled ULTRASOUND IMAGING WITH REDUCED NOISE, filed Apr. 4, 2005, which is hereby incorporated by reference herein in its entirety.

It should be noted that the hemodynamic monitoring system 100 and the various algorithms 200, 400 described herein are particularly adapted for long-term monitoring of patients because the disconnectability makes it possible to remove components of the ultrasound probe 104 from the patient, while still leaving other components in place in the patient for longer periods of time without undue discomfort. The algorithms 200, 400 described herein function synergistically with this hemodynamic monitoring system 100 because it allows for users to both make adjustments for the image quality generated by the ultrasound system 102 and, further, monitor hemodynamic parameters associated with the patient in real-time.

Many of the illustrative examples herein describe the selected anatomical structure as being the left ventricle of a heart; however, the various algorithms and machine learning systems described herein can be trained or otherwise configured to be used with other anatomical structures that are part of the heart (e.g., the right ventricle) or are not associated with the heart without departing from the scope of the present disclosure.

### Noninvasive Hemodynamic Monitoring

When used in combination with each other, the systems and techniques described above allow medical personnel to noninvasively obtain highly accurate quantitative hemodynamic parameters for monitoring a patient. Further, because the hemodynamic parameters being output by the hemodynamic monitoring system 100 are calculated directly from images of the patient's heart, the calculated parameters represent actual quantitative values of the patient's cardiac output and filling volume, rather than mere estimates of these or other hemodynamic parameters that are provided by conventional noninvasive hemodynamic monitoring tools. The aspects of the system described above that provide the functionality are the embodiments of the transesophageal probe described above in combination with the machine learning techniques described above.

In particular, conventional transesophageal echo probes are large and cannot be left in place on the patient for any appreciable length of time (e.g., >20 min). So conventional transesophageal echo systems can provide practitioners with a snap shot of the patient's status, but cannot be used to effectively monitor a patient for an extended period of time, which can be an especially significant drawback for patients that have rapidly changing statuses. Further, conventional ultrasound sensors on the chest surface also cannot be used to monitor patient status for an extended period of time because ultrasound imaging of a patient's heart is heavily operator-dependent (i.e., different users do it in different ways) and, thus, medical teams are not getting the same information in the same way from different users. This likewise means that conventional ultrasound systems cannot be used to effectively monitor a patient for an extended period of time. Conversely, the embodiments of the transesophageal probe described above are miniaturized relative to conventional probes and, further, portions of the transesophageal probe assembly can be detected to improve patient comfort, allowing the probe to be left in the patient for an extended period of time (e.g., up to 72 hours). Therefore, the particular technical aspects of the embodiments of the transesophageal probe allow the probe to be used for hemodynamic monitoring (because it can be left in the patient for extended periods of time relative to conventional ultrasound systems), which in turn allows the machine learning and/or image processing aspects of the hemodynamic monitoring system 100 to receive the necessary input (i.e., images of the patient's heart) to directly calculate the patient's cardiac function and filling parameters in a manner suitable for hemodynamic monitoring (because conventional ultrasound systems could not be used to image the patient for the lengths of time necessary for proper monitoring of the patient).

In sum, the hemodynamic monitoring system 100 described herein allows, for the first time, medical practitioners to receive actual quantitative measurements of the patient's cardiac function and filling in a noninvasive manner that is suitable for monitoring (i.e., instead of just for diagnostic purposes).

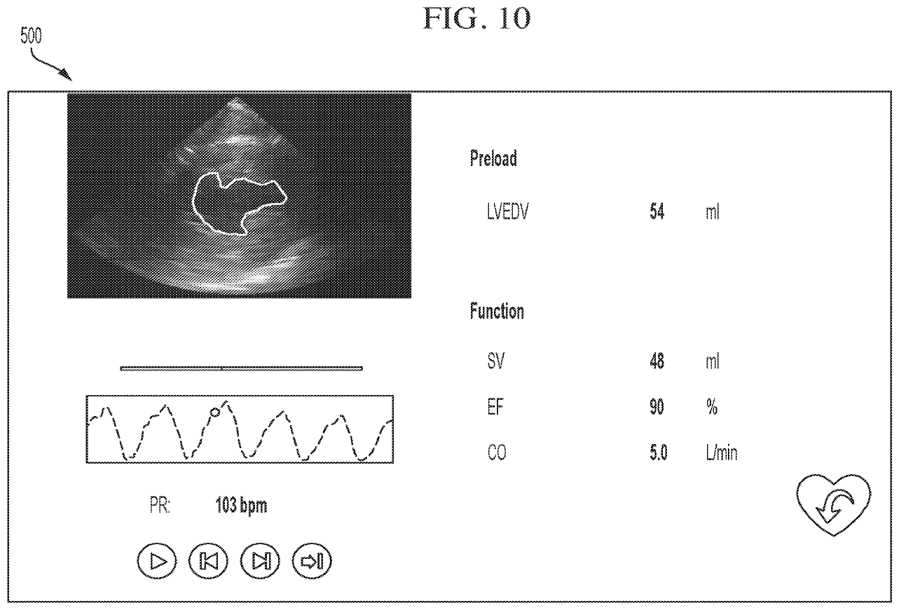

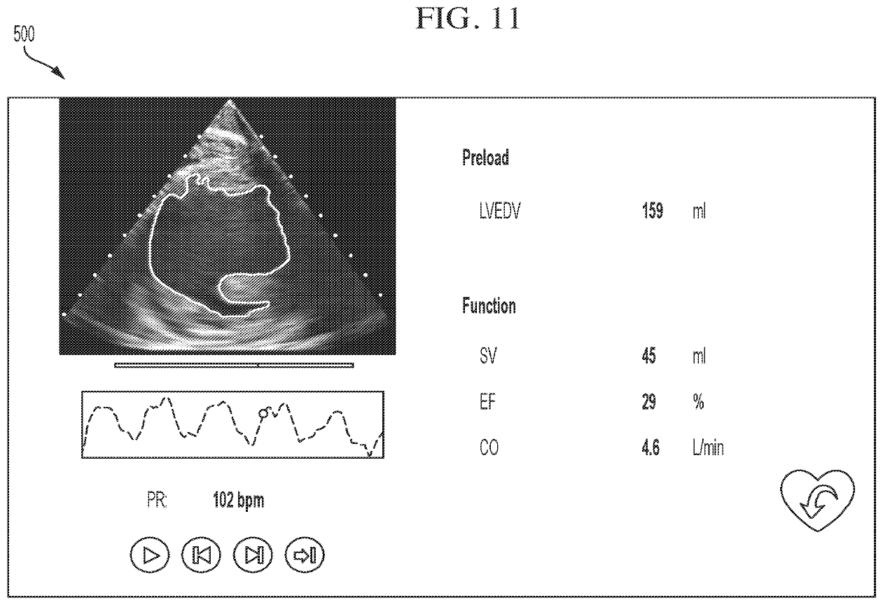

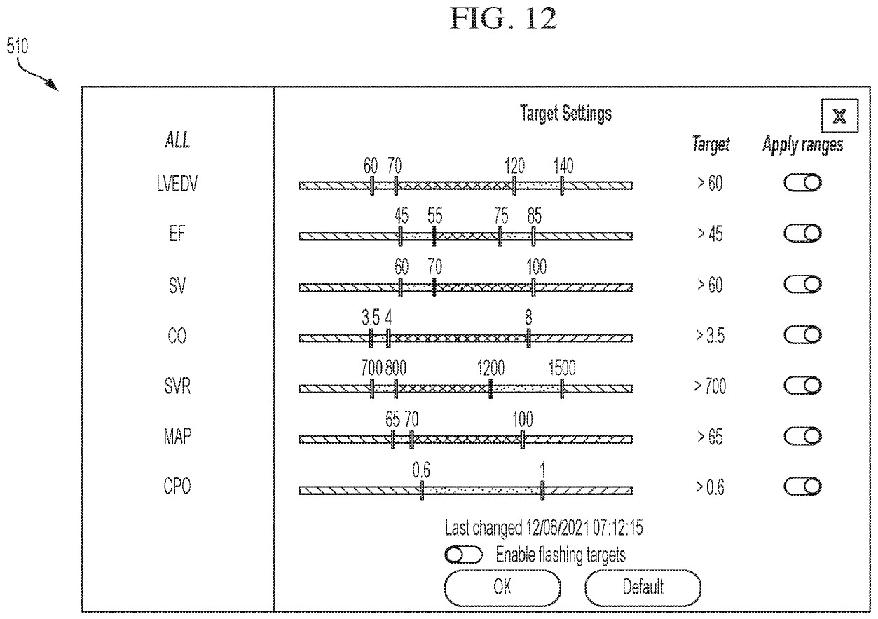

FIGS. 9A-11 illustrate a user interface 500 provided by the hemodynamic monitoring system 100 for sepsis (distributive), hypovolemic, and cardiogenic shock patients, respectively. The user interface 500 could be provided via the display 130, for example. As can be seen, the user interface 500 can display the ultrasound image provided via the ultrasound probe 104, the segmented portion of the image corresponding to the anatomical structure (e.g., the left ventricle of the patient's heart), and various hemodynamic parameters calculated by the computer system 110 therefrom. In one embodiment, such as is shown in FIG. 9A, the hemodynamic parameters provided by the hemodynamic monitoring system 100 could be associated with cardiac flow (i.e., CO), cardiac function (i.e., EF and EV), and cardiac preload (i.e., LVEDV). Notably, LVEDV in particular has never been available to medical practitioners before as a monitoring parameter. Even with the Swan-Ganz catheter, cardiac preload could only be estimated by pulmonary capillary wedge pressure. In another embodiment, such as is shown in FIG. 9B, the hemodynamic parameters provided by the hemodynamic monitoring system 100 could further include SVR and CPO. As can be seen in FIGS. 9A-11, once medical practitioners have access to these hemodynamic parameters and are able to use them for monitoring patients (as opposed to simply seeing snap shots of the patient's current state at any given moment), it becomes much easier for the medical practitioners to diagnose and treat patients and respond appropriately to rapid changes in patients' states.

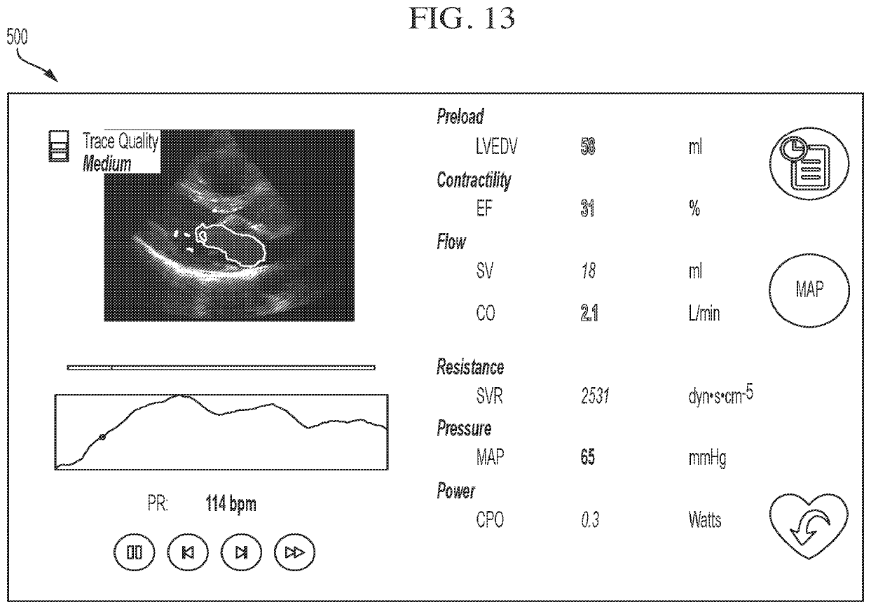

Each patient's response to hemodynamic stress is unique, requiring therapeutic targets that are contextual for each patient's specific health characteristics, disease state, and other situational data. Therefore, it could be highly desirable to allow users to define targets for the various monitored hemodynamic parameters so that medical practitioners can efficiently monitor each patient's unique situation at a glance, without needing to make cumbersome determines on a patient-by-patient basis. In one embodiment, the hemodynamic monitoring system 100 can be further configured to allow for users to set targets and/or endpoints for the various hemodynamic parameters. Further, the hemodynamic monitoring system 100 can be configured to take various actions in response to the monitored hemodynamic parameters relative to the targets and/or endpoints, such as providing prompts or alerts to users. As shown in FIG. 12, the hemodynamic monitoring system 100 can further provide a secondary user interface 510 or screen that allows users to set various targets for some or all of the hemodynamic parameters monitoring by the hemodynamic monitoring system 100. In various embodiments, the targets could include threshold values or ranges. In the embodiment shown in FIG. 12, the user can change the manner in which the hemodynamic parameters are displayed based on where the particular hemodynamic parameter falls within the defined ranges. In other words, the values of the hemodynamic parameters can be displayed with different visual representations (e.g., colors, shapes, or adjacent visual indicators) depending on the particular value relative to the thresholds and/or ranges defined by the input targets. In the embodiment of the user interface 500 illustrated in FIG. 13, the values of the hemodynamic parameters (i.e., LVEDV, EF, SV, CO, SVR, MAP, and CPO) can be shown in different colors depending on where the monitored values fall with respect to the defined targets. This embodiment can be beneficial because it allows medical practitioners to quickly and easily visualize each patient's current hemodynamic parameters relative to expected or target values.

In some embodiments, the hemodynamic monitoring system 100 can be further configured to provide alerts (e.g., visual or audible alerts) depending on the values of the hemodynamic parameters relative to the defined targets. In some embodiments, the hemodynamic monitoring system 100 could be configured to take various additional actions if a patient's hemodynamic parameter values are maintained below or outside of the defined target ranges. For example, if a particular hemodynamic parameter falls below a defined target for a particular period of time, the hemodynamic monitoring system 100 could be configured to provide an alert or prompt for the medical practitioners indicating that an underlying deficit corresponding to the particular hemodynamic parameter needs to be or has not been fully addressed and, accordingly, the medical practitioners should take actions to address the underlying issue. For example, if the patient is losing cardiac volume over a particular length of time, there could be an underlying bleeding issue that must be addressed. As another example, if the patient is toxic over a particular length of time, the patient could be septic. As yet another example, if the patent is ischemic over a particular length of time, there could be an underlying cardiogenic issue that needs to be addressed.

### Use Cases

To further elucidate the concepts and principles of the disclosure described above, a specific use case will be described. This use case is not intended to be limiting in any way, but rather is provided simply to further explain and clarify the benefits of the systems and techniques described herein as compared to conventional hemodynamic monitoring tools.

As generally described above, hemodynamic monitoring and echocardiographic technologies are “siloed” and not able to be effectively used in combination with each other for patient monitoring. This is a well-known issue in the care of circulatory shock patients, as described in Cecconi et al. The problem faced by patients' care teams (which, in the United States, generally consist of a nursing staff member and a care member, i.e., doctor, on rounds) is that circulatory shock is multidimensional and dynamic, which makes it difficult to treat because conventional techniques either provide reliable quantitative hemodynamic output (i.e., the Swan-Ganz catheter), but are highly invasive and cannot be left in the patient for extended periods of time, or do not prove reliable quantitative hemodynamic output. The clinical goal in such a patient is to ensure adequate perfusion, while avoiding fluid overload. However, avoiding fluid overload requires both an echo assessment and hemodynamic monitoring, as described in Cecconi et al. Accordingly, there is a clinical problem (simultaneously monitoring multiple hemodynamic parameters), a technical problem (how to monitor with echocardiography), and an operational problem (the workflow of performing all of these various tasks required by circulatory shock patients). However, the systems and techniques described herein solve the clinical problem and the technical problem by combining both echocardiography (by providing a continuous, extended image stream of the patient's heart) and hemodynamic monitoring into a single hemodynamic monitoring system 100. Accordingly, the hemodynamic monitoring system 100 is able to be effectively utilized by every member of the patient's care team, which in turn solves the operational problem by simplifying the workflow associated with treating the patient's condition.

In particular, the hemodynamic monitoring system 100 described herein could be used in the treatment of circulatory shock patients by first identifying the type of shock (i.e., hypovolemic, distributive and/or septic, cardiogenic, or obstructive) that the patient is suffering from. Second, selecting the appropriate therapeutic interaction using the hemodynamic parameters provided by the hemodynamic monitoring system 100 relative to targets. The therapeutic interventions could include fluids (to increase volume), pressors (to induce vasoconstriction, thereby elevating mean arterial pressure), and/or inotropes (to increase cardiac contractility). Third, evaluating the patient's response to the therapeutic interventions using the hemodynamic monitoring system 100. Conventional tools are not effective for treating patients in this manner because echo cannot effectively be used as a monitoring tool because repeated evaluations of the patient are not feasible. Further, conventional hemodynamic monitoring tools are currently focused on evaluating patients' responses to fluids only (i.e., not pressors and/or inotropes) and, thus, are not effective at monitoring patients' responses to the range of therapeutic interventions available to medical personnel. Because of these limitations of conventional tools, medical staff tend to do trial and error fluid challenges and/or use volume responsiveness as a surrogate target for the patient, which is ineffective and can lead to fluid overload, which is a dangerous and ultimately avoidable complication using the hemodynamic monitoring system 100 described herein.

Referring back to FIGS. 9A-11, it can be seen that the hemodynamic monitoring system 100 can be used to establish hemodynamic profiles (i.e., sets of hemodynamic parameters that fall within various ranges or thresholds) for patients, such as “low preload, high EF.” The hemodynamic profiles can be associated with different types of shock. Accordingly, the hemodynamic monitoring system 100 can be used to identify different types of shock. Without the hemodynamic monitoring system 100, it is very challenging for medical staff members to identify these shock states and monitor the effect of subsequent therapeutic interventions. Therefore, the hemodynamic monitoring system 100 improves the diagnosis and treatment of patients suffering from these various shock conditions, which in turn improves patients' outcomes.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. An hemodynamic monitoring system comprising:
an ultrasound system comprising a transesophageal ultrasound probe configured to obtain a view of a heart; and
a computer system coupled to the ultrasound system, the computer system comprising a processor and a memory, the memory storing instructions that, when executed by the processor, cause the computer system to:
receive a plurality of images of the heart from the ultrasound system obtained via the transesophageal ultrasound probe,
identify, via a first machine learning system trained to identify a region of interest associated with a selected anatomical structure of the heart, the region of interest in the plurality of images,
segment, via a second machine learning system trained to identify the selected anatomical structure using the identified region of interest, a predicted region corresponding to the selected anatomical from the plurality of images based on the identified region of interest, and
calculate, based on the predicted region, a plurality of hemodynamic parameters associated with the heart, the plurality of hemodynamic parameters corresponding to a cardiac function and a cardiac filling.

2. The hemodynamic monitoring system of claim 1, wherein the plurality of hemodynamic parameters comprises at least one of a fractional area change, a heart rate, a stroke volume, a cardiac output, a left ventricular end diastolic volume, or an ejection fraction.

3. The hemodynamic monitoring system of claim 1, wherein the selected anatomical structure of the heart comprises a left ventricle.

4. The hemodynamic monitoring system of claim 1, wherein the machine learning algorithm comprises a convolutional neural network.

5. The hemodynamic monitoring system of claim 1, wherein the memory stores further instructions that, when executed by the processor, cause the computer system to display the plurality of calculated hemodynamic parameters.

6. The hemodynamic monitoring system of claim 1, wherein a first portion of the transesophageal ultrasound probe is configured to be detached from a patient while a second portion of the transesophageal ultrasound probe remains within the patient.

7. The hemodynamic monitoring system of claim 1, wherein the obtained view of the heart comprises at least one of a transgastric short axis view or a mid-esophageal four chamber view.

8. The hemodynamic monitoring system of claim 1, wherein the transesophageal ultrasound probe is configured to be left within a patient for at least 20 minutes.

9. The hemodynamic monitoring system of claim 1, wherein the ultrasound system and the computer system are configured to fit bedside within a patient room of an intensive care unit.

10. The hemodynamic monitoring system of claim 9, wherein the computer system further comprises a display screen configured to display the plurality of calculated hemodynamic parameters at the bedside within the patient room.

11. A hemodynamic monitoring system comprising:
an ultrasound system comprising a transesophageal ultrasound probe configured to obtain a view of a heart; and
a computer system coupled to the ultrasound system, the computer system comprising a processor and a memory, the memory storing instructions that, when executed by the processor, cause the computer system to:
receive a plurality of images of the heart from the ultrasound system obtained via the transesophageal ultrasound probe,
identify a selected anatomical structure associated with the heart from the received plurality of images, and
determine, using a machine learning system trained to output an image quality parameter based on a visualization quality for the selected anatomical landmark in ultrasound images, the image quality parameter for the received plurality of images.

12. The hemodynamic monitoring system of claim 11, wherein the selected anatomical structure of the heart comprises a left ventricle.

13. The hemodynamic monitoring system of claim 11, wherein the machine learning algorithm comprises a convolutional neural network.

14. The hemodynamic monitoring system of claim 11, wherein the memory stores further instructions that, when executed by the processor, cause the computer system to display the calculated quality score.

15. The hemodynamic monitoring system of claim 11, wherein first a portion of the transesophageal ultrasound probe is configured to be detached from a patient while a second portion of the transesophageal ultrasound probe remains within the patient.

16. The hemodynamic monitoring system of claim 11, wherein the obtained view of the heart comprises at least one of a transgastric short axis view or a mid-esophageal four chamber view.

17. The hemodynamic monitoring system of claim 11, wherein the transesophageal ultrasound probe is configured to be left within a patient for at least 20 minutes.

18. The hemodynamic monitoring system of claim 11, wherein the ultrasound system and the computer system are configured to fit bedside within a patient room of an intensive care unit.

19. The hemodynamic monitoring system of claim 18, wherein the computer system further comprises a display screen configured to display the plurality of calculated hemodynamic parameters at the bedside within the patient room.
